(19) United States
(12) Patent Application Publication
(10) Pub. No.: US 2012/0116258 A1
(43) Pub. Date: May 10, 2012

(54) REHABILITATION APPARATUS USING GAME DEVICE

(75) Inventor: Yang-Soo Lee, Daegu (KR)

(73) Assignee: Industry-Acadamic Cooperation Foundation, Kyungpook National University, Daegu (KR)

(21) Appl. No.: 13/244,223

(22) Filed: Sep. 23, 2011

Related U.S. Application Data
(63) Continuation-in-part of application No. 11/387,396, filed on Mar. 23, 2006, now Pat. No. 8,075,449.

(30) Foreign Application Priority Data
Mar. 24, 2005 (KR) 10-2005-24333

(51) Int. Cl. A61B 5/103

(52) U.S. Cl. 600/595

(57) ABSTRACT

An apparatus for lower-limb rehabilitation training of a patient with lower limb paralysis and a rehabilitation apparatus using game device. The invention measures the usage of the paralyzed lower limb, and based upon the measurement, forces patients with limb paralysis to use partially paralyzed lower muscles which they are not likely to use. The apparatus can measure the weight load and the angle of the joint, and by using the measure values as variables, display the condition of paralysis to the patient so that he/she can recognize his/her present condition of paralysis, and enable the user to training through feedback.

### CROSS-REFERENCE TO RELATED APPLICATION

This application is a continuation-in-part application of U.S. patent application Ser. No. 11/387,396, filed Mar. 23, 2006, which claims the benefit of Korean Patent Application No. 10-2005-24333, filed on Mar. 24, 2005 in the Korean Intellectual Property Office, the disclosure of which are incorporated herein by reference.

### BACKGROUND OF THE INVENTION

1. Field of the Invention

The present invention relates to an apparatus and method for lower-limb rehabilitation training of a patient with a paralyzed lower limb (hereinafter will be referred to “patient with lower-limb partial paralysis”), and rehabilitation apparatus using game device. Particularly, the apparatus for lower-limb rehabilitation training of the invention can measure changes in the weight loaded on the lower limb and in the angle of a joint while using the changes in the weight and the joint angle as variables in order to measure the condition of paralysis of the lower limb. In addition, the apparatus of the invention can display the condition of paralysis to the patient with lower-limb paralysis in various ways so that the patient can recognize the usage of the lower limb and make efforts to improve its usage, thereby making feedback effects. Furthermore, by using the rehabilitation training apparatus, the patient can alternatively raise the heels, bend the knees or walk as rehabilitation training.

2. Description of the Related Art

Hemiplegia or partial paralysis (hereinafter will be referred to as “partial paralysis”) originating from apoplexy, traumatic brain damage or cerebral palsy is a motor paralysis that paralyzes muscles or motors so that the muscles paralyzed due to paralysis of one side of a body cannot exert power to a proper extent at necessary moments. In order to treat the patient with partial paralysis, it is possible to use several methods such as physical strengthening, muscle controlling, stretching, balancing and so on. As a rehabilitation training of the patient with upper-limb partial paralysis, a treatment for forcing the patient to use the paralyzed upper limb is being recognized as the most successful among several rehabilitation trainings.

A conventional treatment apparatus for enabling forced use of a paralyzed lower limb is disclosed in for example Korean Patent Application No. 1997-0028382, titled “A Rehabilitation Training System for Balancing a Posture.” As shown in FIG. 1, the system for enabling forced use of the paralyzed lower limb in the document of “A Rehabilitation Training System for Balancing a Posture” includes a part 11 for measuring weight loaded on both lower limbs and a part 12 for notifying measurement values to the user so that the user can compare on which one of the both lower limbs weight is loaded more. However, the prior art of “A Rehabilitation Training System for Balancing a Posture” considers shifting the center of weight only, but does not consider the angle of a joint. Therefore, muscle suffering from further serious paralysis cannot be measured and the contraction of paralyzed muscles cannot be induced. Accordingly, even though the prior art has symmetrically distributed weight, walking ability has not been effectively improved.

In practice, in case of normal walking, one bears the weight on one lower limb with its knee bent for about 15° while raising the other lower limb. Then, the knee of the lower limb supporting the weight is straightened and the weight center of the other lower limb is shifted forward. It is carried out repeatedly. As the normal walking is carried out through the shift of weight center as well as change in the angle of the lower limb, the angle of the joint is an important variable of walking. As illustrated with the normal walking, the shift of the weight center together with change in the angle of the joint enables the movement of the lower limb.

However, a patient with lower-limb partial paralysis has trouble in bending the knee owing to paralysis, and thus tends to shift the center of weight without bending the knee. This results in abnormal walking of the patient.

Accordingly, it should be confirmed whether or not the weight is loaded evenly onto both lower limbs and the joint is systematically working along with the weight load in order that the rehabilitation training system or apparatus for lower-limb partial paralysis can be more effectively used in balance and walking trainings.

Examples of the apparatus for measuring the joint is systematically working along with the weight. Since the three-dimensional walking analysis system three dimensionally analyzes images taken by the three cameras 21 or more, analysis process is complicated and too time-consuming to enable real-time feedback. Furthermore, since the force plate 22 and cameras need a wide place, this system also disadvantageously needs a large space and expensive equipments. Therefore, the three-dimensional walking analysis system is not used for walking training.

Accordingly, there are needs for an apparatus for rehabilitation training that can be constituted of inexpensive equipments to measure the weight load and changes in the joint angle while displaying the measurement in real-time to the user and performing a game using the same.

### SUMMARY OF THE INVENTION

The present invention has been made to solve the foregoing problems of the prior art and it is therefore an object of the present invention to provide an apparatus for lower-limb rehabilitation training that can measure the weight load and the angle of the joint to determine the usage of the paralyzed lower limb thereby enabling lower-limb rehabilitation training based upon the determined usage of the paralyzed lower limb, and a rehabilitation apparatus using game device.

It is another object of the invention to provide an apparatus for lower-limb rehabilitation training that can directly measure the angle of at least one of the knee joint, the ankle joint and the hip joint (i.e., coax) of the lower limb, or indirectly measure the joint angle of the lower limb to detect the usage of the paralyzed muscles thereby enabling rehabilitation training for the paralyzed muscles based upon measurement data, and a rehabilitation apparatus using game device.

It is other object of the invention to provide an apparatus for lower-limb rehabilitation training that can measure the usage of the normal lower limb of a patient with lower-limb partial paralysis to use as reference in measuring the usage of paralyzed muscle or to use normal person's data stored in the apparatus in order to enable normal walking training, and a rehabilitation apparatus using game device.

It is further another object of the invention to provide an apparatus and method for lower-limb rehabilitation training using the weight load and the angle of the joint that can two-dimensionally measure the movement of the joint to rapidly analyze and display the condition of paralysis in order to enable feedback through a process in which the user recognizes his/her condition and endeavors to improve the condition. Furthermore, it is another object of the invention to constitute the apparatus for lower limb rehabilitation training with inexpensive equipments through two-dimensional measurement of the movement of the joint.

Furthermore, it is yet another object of the invention to provide a rehabilitation apparatus using game device that can measure and analyze the usage of the paralyzed muscle of a user who repeatedly carries out task actions with the apparatus for lower limb rehabilitation training attached thereto, and can perform a game using left and right movement of weight and the angle of a joint and perform training through this, so that user can carry out training based upon the measurement and analysis so as to improve the usage of the paralyzed muscle.

In order to realize the above objects, the invention provides a rehabilitation apparatus using game device comprising: a display part to display, for a user, a video game including a graphical game element and a game space in which the graphical game element is configured to perform at least one game action by a command input from the outside; a personal game module including a weight-measuring part being placed below the lower limb to contact with the bottom surface of the lower limb and to measure a value of pressure applied by the lower limb and thereby generate measured weight data, and a joint angle-measuring part to measure a joint angle value of at least one joint of the lower limb and thereby generate measured joint angle data, and the command for the game action of the game element; and a controller to generate the video game required to vary the weight applied to the lower limb and the joint angle of the lower limb, to control a first game action of the graphical game element of the video game based on a change in the measured value of pressure, and to control a second game action of the graphical game element of the video game based on a change in the measured joint angle value of the lower limb.

The joint angle-measuring part may include an electric goniometer attached to at least one joint, and include at least one of a knee joint goniometer for measuring the angle of a knee joint, an ankle joint goniometer for measuring the angle of an ankle joint, and a hip joint goniometer for measuring the angle of a hip joint. One of ordinary skill in the arts will understand that other joint angle measuring devices than the goniometer can be used in accordance with this disclosure.

Here, the joint angle-measuring part may include all of the knee joint goniometer, the ankle joint goniometer, and the hip joint goniometer in order to synthetically measure whether the lower limb is moved using major muscles of the lower limb including thigh muscles and calf muscles, and whether there is a force for raising the lower limb.

Also, the joint angle-measuring part may include at least one of a tilting sensor or a gyro sensor attached to the lower leg or the thigh, an accelerometer attached to a knee joint, a magnetic sensor for measuring a distance between a reference point and a predetermined region of a body of the user, and an ultrasonic distance measuring device, in order to indirectly measure a reduction and an expansion in the joint angle of the lower limb.

The first game action may be a movement of the graphical game element into one direction and a movement into an opposite direction thereto in the game space.

Meanwhile, at least two personal game modules may be provided, each personal game module may be connected to a single controller, and each of at least two users may be enabled to play the same game by individually employing the personal game module.

Also, to realize the above objects, the invention provides a rehabilitation apparatus using game device comprising: a display part to display, for a user, a video game including a graphical game element and a game space in which the graphical game element is configured to perform at least one game action by a command input from the outside; a personal game module including a weight-measuring part being placed below the lower limb to contact with the bottom surface of the lower limb and to measure a value of pressure applied by the lower limb and thereby generate measured weight data, and an angle-measuring part to measure an angle value of the lower limb and thereby generate measured angle data, and to input the command for the game action of the game element; and a controller to generate the video game required to vary the weight applied to the lower limb and the joint angle of the lower limb, to control a first game action of the graphical game element of the video game based on a change in the measured value of pressure, and to control a second game action of the graphical game element of the video game based on a changed in the measured angle value of the lower limb.

The angle-measuring part may include an electric goniometer attached to at least one joint, and include at least one of a knee joint goniometer for measuring the angle of a knee joint, an ankle joint goniometer for measuring the angle of an ankle joint, and a hip joint goniometer for measuring the angle of a hip joint.

Here, the angle-measuring part may include all of the knee joint goniometer, the ankle joint goniometer, and the hip joint goniometer in order to synthetically measure whether the lower limb is moved using major muscles of the lower limb including thigh muscles and calf muscles, and whether there is a force for raising the lower limb.

The angle-measuring part may include at least one of a tilting sensor or a gyro sensor attached to the lower leg or the thigh, an accelerometer attached to a knee joint, a magnetic sensor for measuring a distance between a reference point and a predetermined region of a body of the user, and an ultrasonic distance measuring device, in order to indirectly measure a reduction and an expansion in the joint angle of the lower limb.

The first game action may be a movement of the graphical game element into one direction and a movement into an opposite direction thereto in the game space.

Meanwhile, at least two personal game modules may be provided, each personal game module may be connected to a single controller, and each of at least two users may be enabled to play the same game by individually employing the personal game module.

Also, to realize the above objects, the invention provides a rehabilitation apparatus using game device comprising: a display part to display, for a user, a video game including at least one graphical game element and a game space in which the graphical game element is configured to perform at least one game action by a command input from the outside; a weight-measuring part being placed below the lower limb to contact with the bottom surface of the lower limb and to measure a value of pressure applied by the lower limb and thereby generate measured weight data; a joint angle-measuring part to directly measure a joint angle value of at least one joint of a knee joint, an ankle joint, and a hip joint of the lower limb or indirectly measure the joint angle of the lower limb and thereby generate measured joint angle data; and a controller to generate the video game required to vary the weight applied to the lower limb and one joint angle of the lower limb, to control a first game action of a first graphical game element of the video game based on a change in the measured value of pressure, and to control a second game action of a second graphical game element of the video game based on a change in the measured joint angle value.

The joint angle-measuring part may include at least one of a knee joint goniometer for measuring the angle of a knee joint, an ankle joint goniometer for measuring the angle of an ankle joint, and a hip joint goniometer for measuring the angle of a hip joint.

Also, the joint angle-measuring part may include at least one of a tilting sensor or a gyro sensor attached to the lower leg or the thigh, an accelerometer attached to a knee joint, a magnetic sensor for measuring a distance between a reference point and a predetermined region of a body of the user, and an ultrasonic distance measuring device, in order to indirectly measure a reduction and an expansion in the joint angle of the lower limb.

The first graphic game element and the second graphic game element may be the same game element, and the first game action and the second game action may be different from each other.

The first graphic game element may be a character and the second graphic game element may be a game element operated by the character.

Also, the first game action may be a movement of the graphical game element into one direction and a movement into an opposite direction thereto in the game space, or may be to adjust a size of the graphic game element in the game space.

Also, the controller may generate movement amount data of the user as a result of the video game to display the movement amount data on the display part.

### BRIEF DESCRIPTION OF THE DRAWINGS

The above and other objects, features and other advantages of the present invention will be more clearly understood from the following detailed description taken in conjunction with the accompanying drawings, in which:

FIG. 1 illustrates a conventional apparatus for rehabilitation training using weight load and its user;

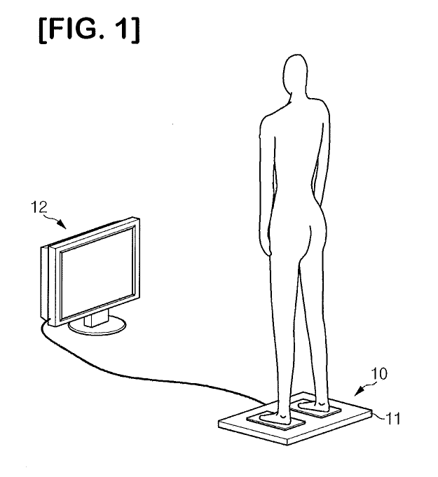

FIG. 2 illustrates a conventional three-dimensional walking analysis system using three cameras and a force plate;

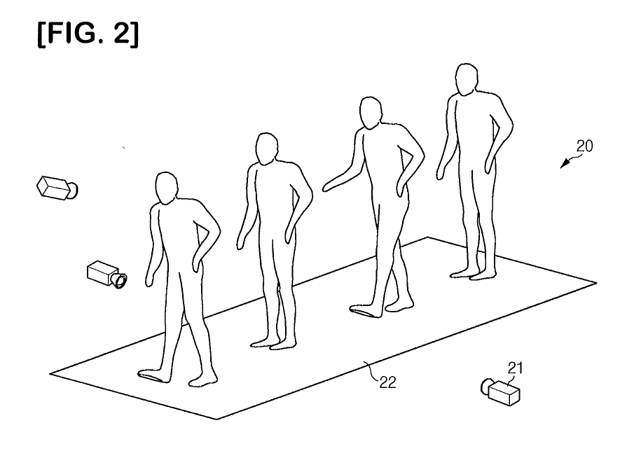

FIG. 3 is a block diagram illustrating an apparatus for lower-limb rehabilitation training of the invention;

FIGS. 4a and 4b illustrate a force plate and a balance as a weight-measuring part;

FIG. 5 illustrates an electric goniometer as a joint angle-measuring part;

FIG. 6 illustrates a variable resistance goniometer as a joint angle-measuring part;

FIG. 7 illustrates electric goniometers used at the ankle joint and the hip joint and a variable resistance goniometer used at the knee joint;

FIG. 8 illustrates a motion-capturing goniometer used as a joint angle-measuring part;

FIG. 9 illustrates a tilting sensor used as a joint angle-measuring part;

FIG. 10 illustrates an accelerometer used as a joint angle-measuring part;

FIG. 11 illustrates an ultrasonic distance measuring device used as a joint angle-measuring part;

FIG. 12 is a flowchart illustrating a process executed by a controller;

FIG. 13 is a diagram illustrating a screen of a display part;

FIG. 14 illustrates a balance training of a user;

FIG. 15 illustrates a standing training of a user;

FIG. 16 illustrates a walking training of a user;

FIG. 17 is a diagram illustrating a configuration of a rehabilitation apparatus using game device according to an embodiment of the invention;

FIG. 18 is a diagram illustrating an example in which an embodiment of a game configured by a rehabilitation apparatus using game device according to an embodiment of the invention is displayed on a display part; and

FIG. 19 is a diagram illustrating an example in which another embodiment of a game implemented by a rehabilitation apparatus using game device according to an embodiment of the invention is displayed on a display part.

### DESCRIPTION OF THE PREFERRED EMBODIMENTS

The detailed description will present preferred embodiments of the invention with reference to the accompanying drawings.

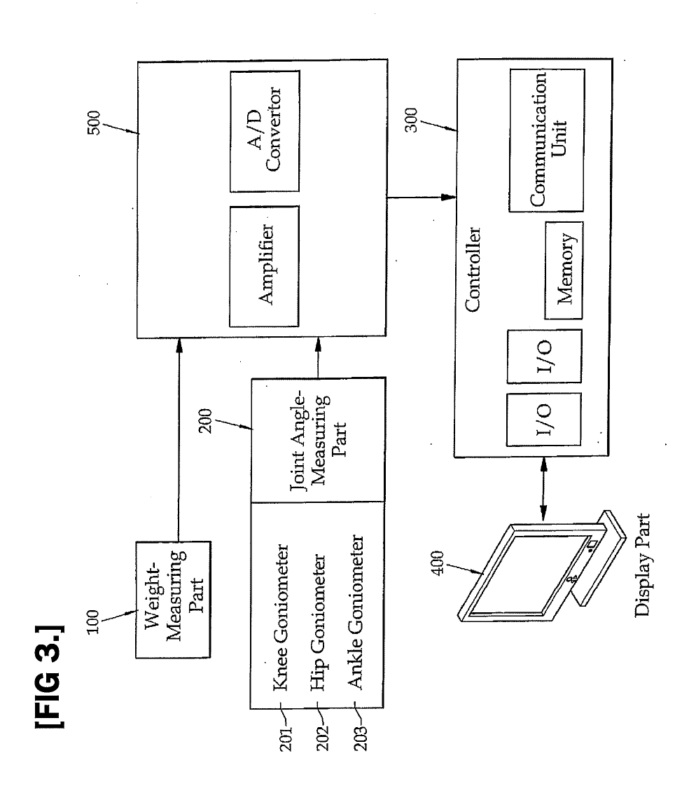

FIG. 3 is a block diagram illustrating an apparatus for lower-limb rehabilitation training using weight load and joint angle of the lower limb according to the invention. As shown in FIG. 3, the apparatus for lower-limb rehabilitation training includes a weight-measuring part 100, a joint angle-measuring part 200, a controller 300 and a display part 400.

The weight-measuring part 100 is a part for measuring weight load that varies according to user motion, and disposed at a position contacting the bottom of a lower limb in order to measure the weight loaded on a lower limb. The weight-measuring part 100 may be provided to measure only one lower limb, which may be a paralyzed one. Of course, the weight-measuring part 100 can be configured to measure the weight loaded on both lower limbs.

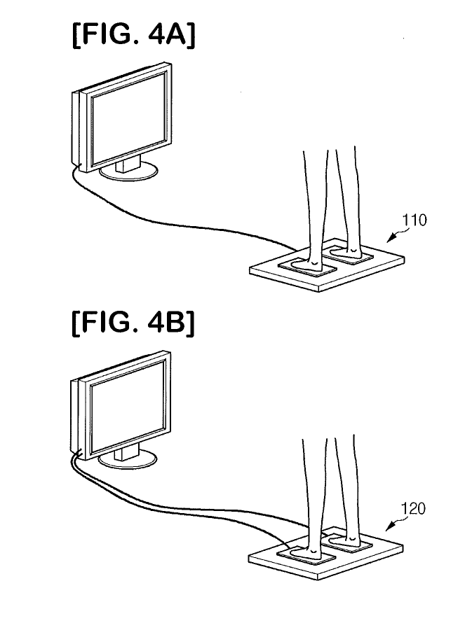

As shown in FIG. 4a, a force plate 110 including at least one pressure sensor that contacts directly or indirectly the bottom of the lower limbs may be used as the weight-measuring part 100. The pressure sensor can change its own status in response to the pressure, such that the weight loaded on the lower limbs can be measured based upon the changed status of the pressure sensor. In addition, at least one sensor may be attached to the bottom of each lower limb in order to measure the weight loaded to the lower region of the each lower limb.

As shown in FIG. 4b, a balance 120 including an elastic mechanism that directly or indirectly contacts a lower region of the lower limb, an electronic balance using a load cell, and a strain gauge can be adopted as the weight-measuring part 100. The elastic mechanism of the balance 120 can vary its status under the weight loaded onto the lower limb in order to measure the weight loaded onto the lower limb. The varying status of the elastic mechanism may include but not limited to the volume or length.

The joint angle-measuring part 200 may directly or indirectly measure the joint angle of the lower limb.

The joint angle-measuring part 200 according to a scheme of directly measuring the joint angle of the lower limb comprises at least one of a knee joint goniometer 201 for measuring the angle of a knee joint, a hip joint goniometer 202 for measuring the angle of a hip angle and an ankle joint goniometer 203 for measuring the angle of an ankle joint.

Here, the joint angle-measuring part 200 may use an electric goniometer 210, a variable resistance goniometer 220 and a motion capture goniometer 230 in order to measure the angle of joints (e.g., knee joint, ankle joint and hip joint).

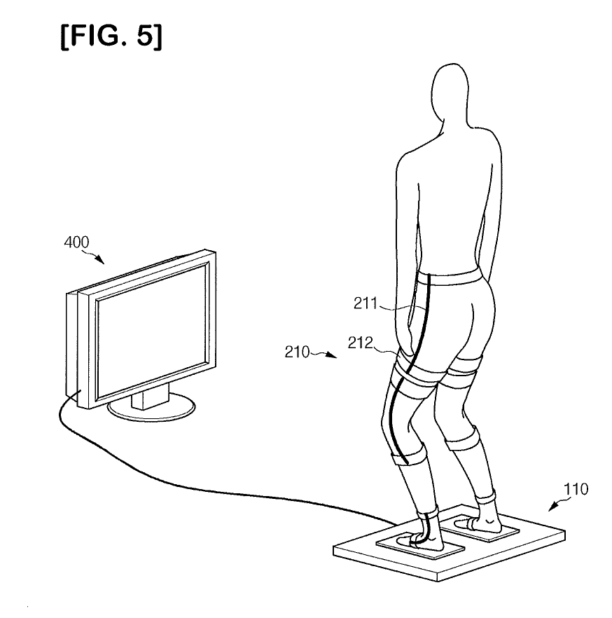

FIG. 5 shows the electric goniometer 210 usable for the joint angle-measuring part 200. As shown in FIG. 5, the electric goniometer 210 includes a flexible bar 211 made of an elastic material so as to flex along with the bending of the joint and an attachment support 212 connected to a region of the lower limb and the flexible bar 211 for allowing the flexible bar 211 to flex along with the joint. The attachment support 212 of the electric goniometer 210 is attached to upper and lower regions of the joint the angle of which is to be measured, and connected to the flexible bar 211 which is oriented in the same direction of the joint.

In the knee joint goniometer 201, the attachment support 212 is disposed along the thigh and the calf. The flexible bar 211 is connected to the attachment support 212, and oriented in the same direction along which the knee is to bend. In this way, the angle of the knee joint is measured based upon the degree of bending of the flexible bar 211 that flexes along with the knee joint. In the ankle joint goniometer 203, the attachment support 212 is disposed on the calf and the foot so that the flexible bar 211 connected to the attachment support 212 flexes along with changes in the angle of the ankle. In this way, the angle of the ankle is measured based upon the bending of the flexible bar 211. Also in the hip joint goniometer 202, the attachment support 212 is attached to the waist and the thigh and the flexible bar 211, which is oriented to the same direction of the hip joint changing its angle in response to the leg being raised, is connected to the attachment support 212 in such a fashion that the angle of the hip joint is measured based upon the degree of bending of the flexible bar 211.

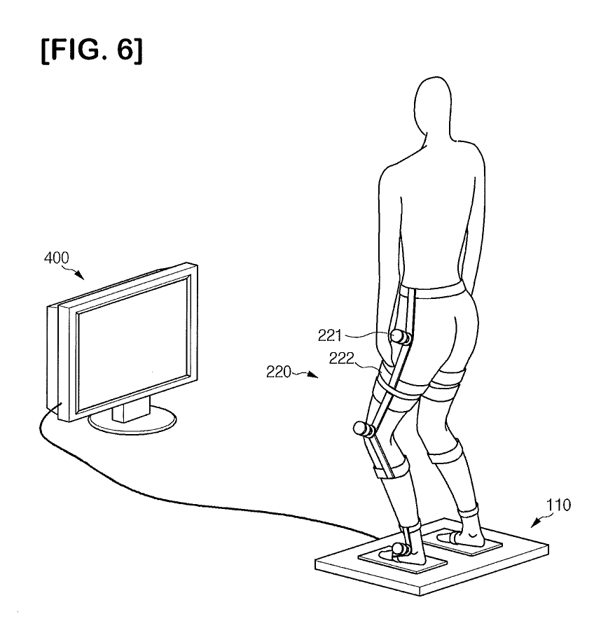

FIG. 6 illustrates the variable resistance goniometer 220 used as the joint angle-measuring part 200. As shown in FIG. 6, the variable resistance goniometer 220 includes a variable resistor 221, which is variable in resistance according to the angle of the joint, and an attachment support 222 for fixing the variable resistor 221 to the lower limb. Like the attachment support 212 of the electric goniometer 210, the attachment support 222 is placed on the thigh and the calf in case of the knee joint goniometer 201, on the thigh and the foot in case of the ankle joint goniometer 203, and on the waist and the thigh in case of the hip joint goniometer 202. The variable resistor 221 of the variable resistance goniometer 220 has a resistance adjuster that is provided to move along with the joint so that the resistance of the variable resistor is varied according to changes in the angle of the joint. In this way, it is possible to measure the angle of the joint based upon the resistance varying in response to the angle of the joint.

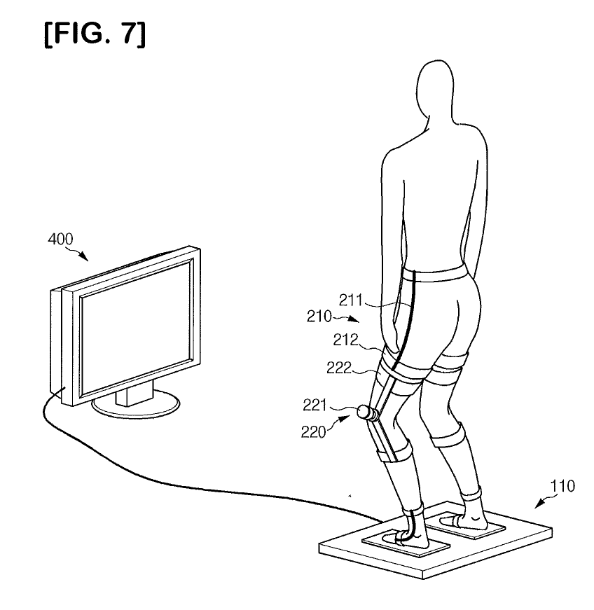

Furthermore, as shown in FIG. 7, the joint angle goniometer 200 may adopt different goniometer types according to the knee, ankle and hip joints. For example, the knee joint goniometer 201 may adopt the variable resistance goniometer 220, and the ankle and hip joint goniometers 203 and 202 may adopt the electric goniometer 210. However, these are illustrative only, but the joint goniometer 220 may adopt any combination of goniometers.

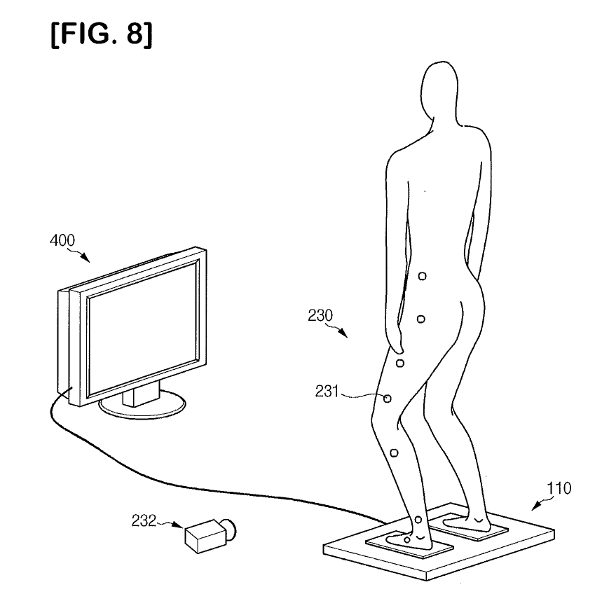

FIG. 8 illustrates the motion capture goniometer 230 used as the joint angle-measuring part 200. The motion capture goniometer 230 includes patches 231, which are attachable to the joint of the lower limb and limb regions above and under the joint, and a camera 232 that can detect the position of the patches 231. Then, the joint angle-measuring part 200 can detect the position of the patches 231 attached to the lower limb, varying in response to the movement of the lower limb by using the camera 232, and thus measure the angle of the joint through the varying position of the patches 231. The position of the patches 231 may be varied according to the knee, ankle and hip joint goniometers. For example, in the knee joint goniometer 201, the patches 231 are attached to the thigh and the calf, which are regions above and under the knee ankle. In the hip joint goniometer 202, the patches 231 are attached to the hip joint, the waist and the thigh. Furthermore, the patches 231 are preferably attached to those regions facing the camera 232 so that they can observe the patches 231. More preferably, the patches 231 are attached to side regions of the lower limb so that the camera 231 can measure the angle varying in response to the movement of the joint when the lower limb is raised or the knee is bent.

In the meantime, the joint angle-measuring part 200 according to a scheme of indirectly measuring the joint angle of the lower limb may use a tilting sensor, a gyro sensor, an accelerometer, a magnetic sensor, an ultrasonic distance measuring device, and the like.

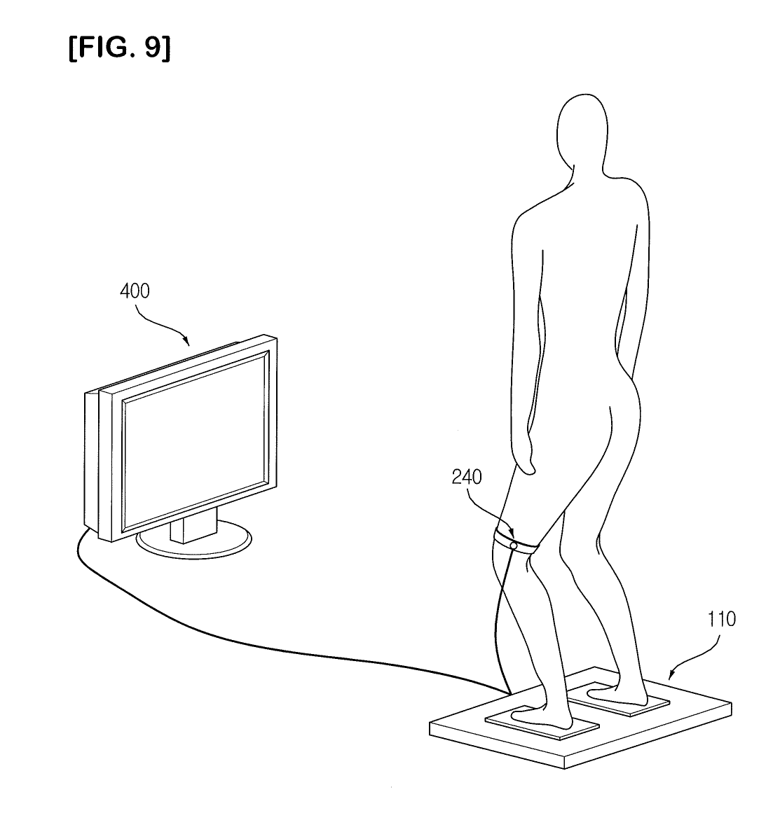

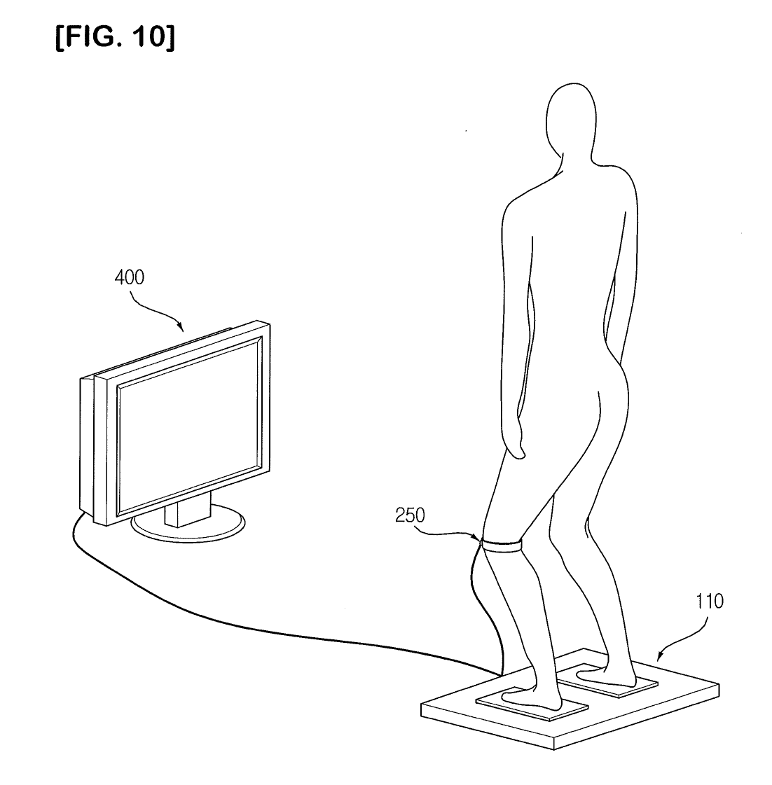

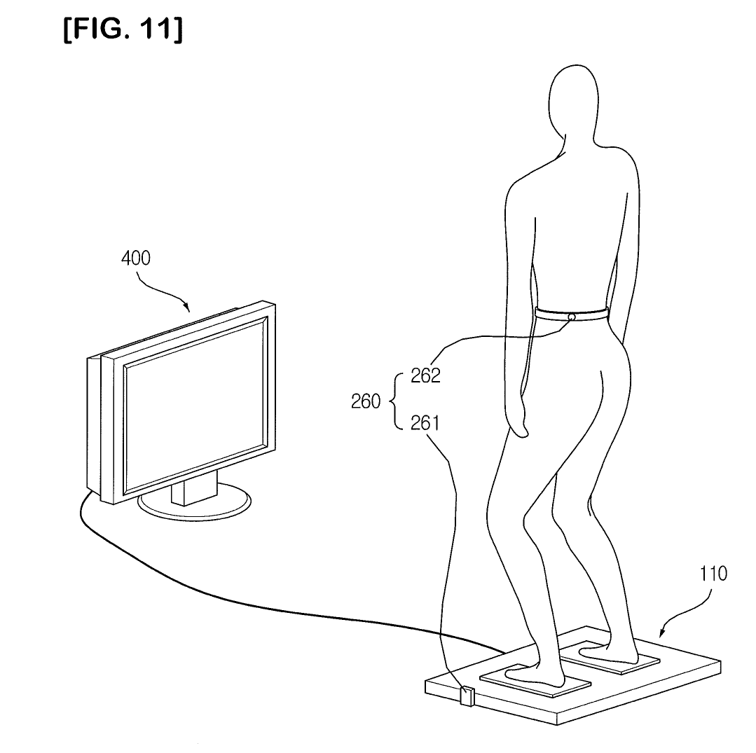

FIG. 9 illustrates a tilting sensor used as a joint angle-measuring part, FIG. 10 illustrates an accelerometer used as the joint angle-measuring part, and FIG. 11 illustrates an ultrasonic distance measuring device used as the joint angle-measuring part.

Referring to FIG. 9, the joint angle of the lower limb may be indirectly measured by measuring the slope of the thigh or the lower leg using the tilting sensor 240 or the gyro sensor. For this, the tilting sensor 240 or the gyro sensor may be attached to one region of the thigh or the lower leg. When a user bends or straightens a joint of the lower limb in a state where the tilting sensor 240 or the gyro sensor is attached to the thigh or the lower leg, the slope of the attachment region may be measured by the tilting sensor 240 and the gyro sensor and the joint angle of the lower limb may be calculated using the measurement value.

Referring to FIG. 10, the joint angle of the lower limb may be indirectly measured by attaching the accelerometer to one region of the lower limb and measuring the acceleration of the lower limb. The accelerometer may be attached to various regions of the lower limb, however, preferably to the front of a knee joint. The knee joint moves forward when the lower limb is bent, and moves backward when the lower limb is straightened. Therefore, the joint angle of the lower limb may be indirectly measured by measuring the acceleration of the knee joint using the accelerometer.

Also, in a state where feet of the user touch the floor, if the user bends the lower limb, a body of the user downwardly moves, and if the user straightens the lower limb, the body of the user upwardly moves. Therefore, the joint angle of the lower limb may be indirectly measured by measuring a distance between a reference point and a predetermined region of the body of the user using the magnetic sensor and the ultrasonic distance measuring device.

Specifically, to indirectly measure the angle of the joint using the magnetic sensor, a permanent magnet is attached to the predetermined region of the user body, for example, a waist and the magnetic sensor is attached to the outer side of the partially paralyzed lower limb. Since a distance between the permanent magnet and the magnetic sensor becomes close when bending the corresponding lower limb, the magnetic force increases. On the contrary, when straightening legs, the distance between the magnet and the magnetic sensor becomes distant, the magnetic force decreases. It is possible to measure the absolute distance between the magnetic sensor and the permanent magnet using a change in the magnitude of the magnetic force, or to measure the relative movement between the magnetic sensor and the permanent magnet.

Even when using the ultrasonic distance measuring device as the joint angle-measuring part, the joint angle of the lower limb may be indirectly measured in a similar manner. Referring to FIG. 11, when the user bends or straightens the lower limb in a state where an ultrasonic generator 261 is installed on the floor and an ultrasonic reflector 262 is attached to a predetermined region of the user, for example, a waist, a distance between the ultrasonic generator 261 and the ultrasonic reflector 262 becomes close or distant. Therefore, a change in the joint angle of the lower limb may be indirectly measured by measuring the distance that is measured by the ultrasonic distance measuring device 260 based on the change in the joint angle of the lower limb of the user.

In addition, the apparatus for lower-limb rehabilitation training of the invention may also include an amplifier-converter 500 for converting data, which is measured in real-time by the weight-measuring part 100 and the joint angle-measuring part 200, into an electric signal. While the amplifier-converter 500 may be included in the weight-measuring part 100 and the joint angle-measuring part 200, it can be provided in the controller 300 or as a separate part.

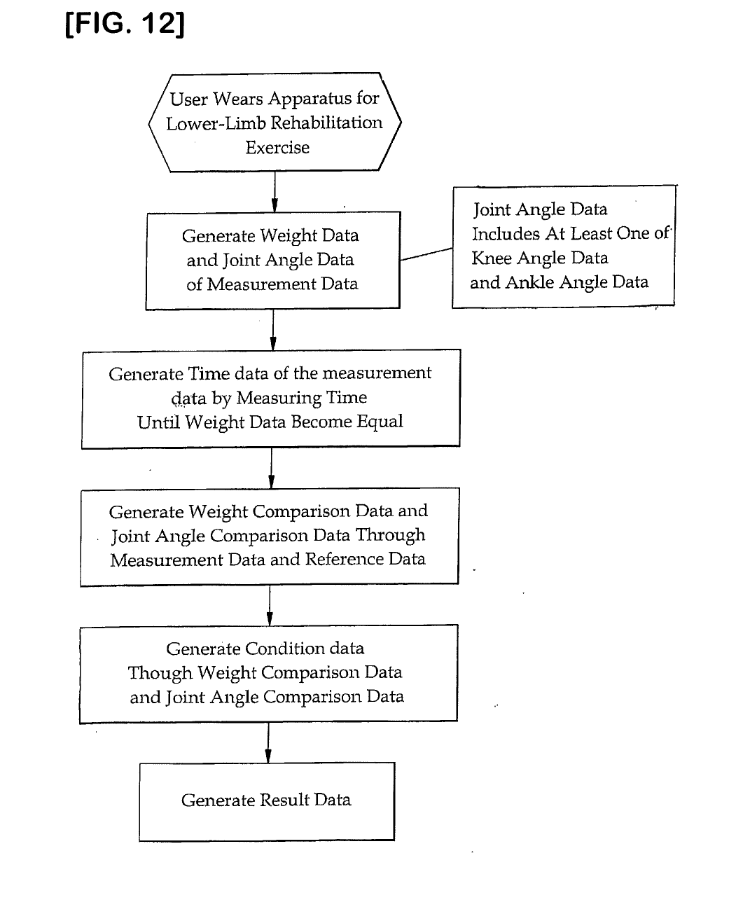

In the apparatus for lower-limb rehabilitation training of the invention, based upon measurement data obtained from the weight-measuring part 100 and the joint angle-measuring part 200, the controller 300 generates result data to be displayed by the display part 400. FIG. 12 is a flowchart illustrating a process by the controller 300 for generating result data.

The controller 300 reads measurement values simultaneously from the weight-measuring part 100 and the joint angle-measuring part 200 or controls the weight-measuring part 100 and the joint angle-measuring part 200 to simultaneously implement measurement. The controller 300 also generates the value read from the weight-measuring part 100 into weight data of measurement data, and the value read from the joint angle-measuring part 200 into measured joint angle data. Since the joint angle-measuring part 200 includes at least one of the knee joint goniometer, the ankle joint goniometer 203 and the hip joint goniometer, the controller 300 accordingly generates the measurement data as knee angle data, ankle angle data and hip angle data.

In addition, the controller 300 counts time until the weight data of the measurement data becomes substantially the same value, and generates the counted time as time data of measurement data. The time data of the measurement data is generated since the time required for the paralyzed lower limb to move may be different from the time required for the normal lower limb to move. For example, since it is more difficult to move the paralyzed lower limb and walking requires more effort, the paralyzed lower limb may move in a shorter pace or take more time per step in walking. For this reason, the apparatus for lower-limb rehabilitation training of the invention measures the time elapsed during the movement of the paralyzed lower limb to generate measurement time data so that the measurement time data can be compared with reference time data required for a normal lower limb to move. Then, the controller generates time comparison data from the difference between the measurement time data and the reference time data.

The reference data functions as a reference with which the measurement data is to be compared, and includes at least one of weight data, knee angle data, ankle angle data, hip angle data and time data like the measurement data including at least one of weight data, knee angle data, ankle angle data, hip angle data and time data. The reference data may be generated through the measurement of the lower limb of normal people similar to previously stored basic information of the user, or through estimation. Otherwise, the reference data may be measured in real-time from the normal lower limb of the patient having the paralyzed lower limb, from which the measurement data are generated. If the measurement data is taken in real-time from the normal lower limb, in order to compare the conditions of the both lower limbs in the same position, it is preferable that one is selected from the measurement data and the reference data, and the value of the selected data, which is taken half-period before the present time, is compared with the present value of the unselected data.

The controller 300 compares the weight and joint angle data of the measurement data with the weight and joint angle data of the reference data to generate weight and joint angle comparison data. The joint angle data includes at least one of the knee angle data, the ankle angle data and the hip angle data as described above, and accordingly the joint angle comparison data includes at least one knee angle comparison data, ankle angle comparison data and hip angle comparison data.

In addition, when the measurement data is compared with the reference data in order to generate the weight comparison data and the joint angle comparison data, it is enough to confirm whether or not the measurement data and the reference data are similar. So, subtraction and/or division can be selectively used. Subtraction generates comparison data from the difference, and division generates comparison data from the ratio. It is apparent that the invention can use at least one of the above-described methods in order to generate the comparison data.

Then, through the weight comparison data and the joint angle comparison data, the controller 300 generates condition data numerically informing the usage of paralysis. The condition data can be produced from the weight comparison data with the joint angle comparison data by addition or multiplication. These operations for producing the condition data are illustrative only, but other operations may be adopted to produce the condition data.

If the condition data is within an error range, the controller 300 generates a message informing that the usage of lower limb muscle or its rehabilitation training is normal. The condition error can be set according to the user, or used as values previously stored in the apparatus. The condition error can be varied according to operations for generating the condition data.

Based upon the condition data, the weight comparison data and the joint angle comparison data, it is possible to generate a message available for feedback training of the user.

If the condition data is out of the error range, in order to judge which part of the paralyzed lower limb disables the lower limb from normally moving, first the weight comparison data is detected, and if the weight comparison data is within the error range, the joint angle comparison data is detected. This order of detection may be set different according to the user, training purpose or training method. That is, the weight comparison data can be detected after the detection of the joint angle comparison data. In case of balance or standing training, the weight comparison data will be dominant in the condition data since a patient with lower-limb paralysis is unlikely to strain the paralyzed lower limb. However, in case of walking training where the total weight is repeatedly concentrated on one lower limb and then on the other lower limb, the condition data is influenced unlikely by the weight data but likely by the joint angle comparison data. It is highly probable that the condition data can be out of the error range by the joint angle comparison data. Thus, it is preferable that the joint angle comparison data is first detected for the purpose of rapid process.

If the weight comparison data is out of the error range, there is generated a message instructing that the weight load be further biased to the lower limb under the measurement or reduced according to the importance of the reference data and the measurement data.

The joint angle comparison data will be detected as follows. In detection of the joint angle comparison data, if all of the knee, ankle and hip angles are measure, the knee angle comparison data is detected first, and if the knee angle comparison data is within the error range, a message is generated with respect to a bigger one of the ankle angle comparison data and the hip angle comparison data. If the knee angle comparison data is out of the error range, the controller 300 generates a message instructing that the knee be further bent or straightened according to the importance of the measurement data and the reference data.

If the knee angle comparison data is within the error range of the knee angle, a larger one is selected from the ankle and hip angle comparison data as described above. If the ankle angle comparison data is larger than the hip angle comparison data, there is generated a message instructing movement of the ankle with further or less bending thereof according to the importance of the ankle angle data of the reference data and the ankle angle data of the measurement data. If the knee angle comparison data is smaller than the hip angle comparison data, there is generated a message instructing movement of the leg with further raising or less raising thereof according to the importance of the hip angle data of the reference data and the hip angle data of the measurement data.

Then, the controller 300 generates a result data to be transmitted to the display part. The result data includes at least one selected from the group consisting of time data, weight data, knee angle data, ankle angle data and hip angle data of the measurement data; time data, weight data, knee angle data, ankle angle data and hip angle data of the reference data; weight comparison data, knee angle comparison data, ankle angle comparison data, hip angle comparison data, time comparison data and condition data. The result data may also include a message that is so generated for allowing the user to suitably training.

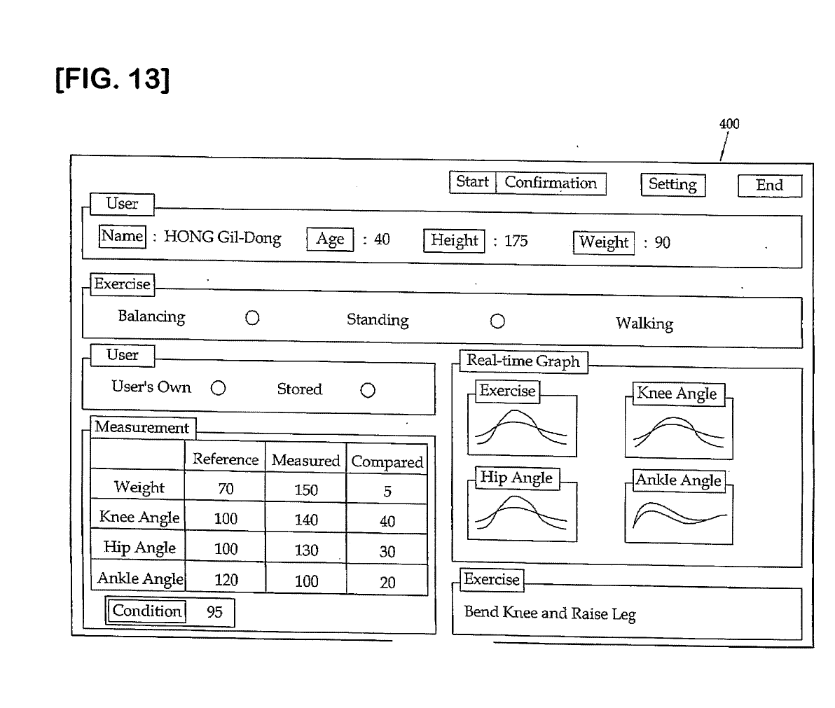

The apparatus for lower-limb rehabilitation training of the invention includes the display part 400 for displaying result data generated from the controller 300 to the user. As shown in FIG. 13, examples of the display part 400 may include a dedicated monitor, a display board, an LED window, a general computer monitor and so on that can be used to display contents to the user in the apparatus for lower-limb rehabilitation training.

The display part 400 displays basic information including name, age, weight and height of the user and result data generated by the controller. Furthermore, the display part 400 can use characters or sign lamps to display messages associated with user condition of the result data generated by the controller 300 so that the user can easily notice. This is an example only, but the invention is not limited thereby.

In displaying the result data generated by the controller 300, the display part 400 can plot a graph with time on one axis and data on the other in order to assist the user to recognize the usage of the paralyzed muscle.

In case of the weight data graph, the weight data of the reference data and the weight data of the measurement data measured in real-time are displayed on the graph with time on one axis and the weight data on the other axis. The user can easily recognize whether or not two data are similar, and can training the paralyzed lower limb in order to make the two data similar.

In case of a knee angle data graph, the knee angle data of the reference data and the knee angle data of the measurement data, measured in real time, are displayed on the graph with time on one axis and the knee angle data on the other axis. Hip and ankle angle data graphs are plotted similar to the above-described knee angle data graph.

Furthermore, the condition data may be displayed on a graph with the condition data, which are generated through the weight comparison data and the joint angle comparison data, on one axis and time on the other axis. The slope of the condition data graph will decrease as the user trainings properly.

Of course, different processes may be adopted to generate the graphs in condition that they comply with the purpose of allowing the user to easily notice his/her condition in real-time.

In addition, the result data can be expressed by numbers or characters other than the graphs. A data having the largest value of the condition data, the weight comparison data and the joint angle comparison data can be displayed via an LED so that the user can easily recognize a region that should be most trained in rehabilitation of paralysis.

The contents expressed by the numbers, characters and graphs may be varied whenever data are newly measured and added.

Also, the invention may provide a rehabilitation apparatus using game device that can generate a game using the result data as a game variable so that the user can be interested in training.

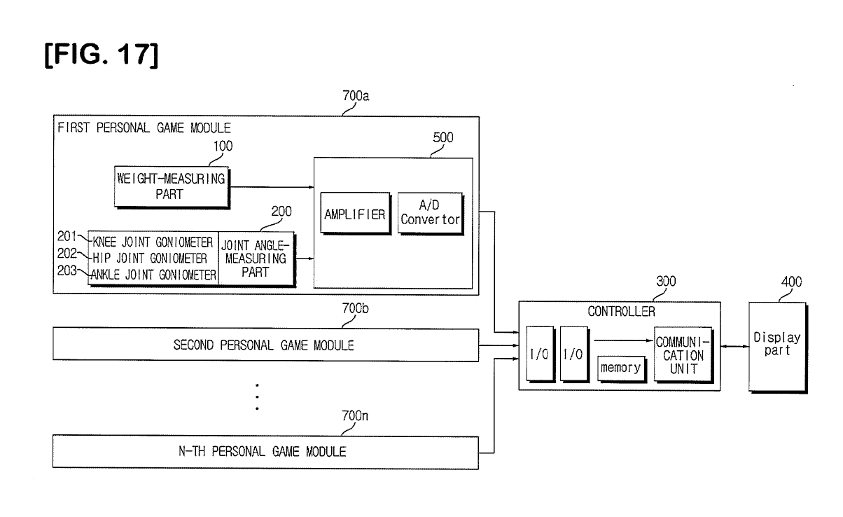

Referring to FIG. 17, a rehabilitation apparatus using game device according to an embodiment of the invention includes a personal game module 700, a controller 300, and a display part 400.

The personal game module 700 is an input means for inputting a predetermined command for execution of a game by sensing a training motion of the user. The personal game module 700 includes a weight-measuring part 100 and a joint angle-measuring part 200.

As described above, the weight-measuring part 100 measures weight load that varies based on each movement of the user, and the joint angle-measuring part 200 measures the joint angle of the lower limb. Therefore, the weight-measuring part 100 and the joint angle-measuring part 200 may be an input means for playing a game.

A game program that the user plays is stored in the controller 300. At least two game programs may be stored in the controller 300. The user may select and play any one of the stored game programs. A game to be stored in the controller 300 may be an existing game and may also be a new game that has not existed. Depending on necessity, a new game may be added to the controller 300 or an existing game may be deleted. The controller 300 collects data of the user that is measured by the weight-measuring part 100 and the joint angle-measuring part 200, generates result data using the collected data, drives the game program by using the result data as a game variable, and transfers output information of the game program to the display part 400.

The display part 400 outputs, as an image, the game information generated by the controller 300, and displays the image for the user.

The personal game module 700 of the rehabilitation apparatus using game device of the invention may additionally include an amplifier and a converter 500 to convert, to an electrical signal, data that is measured in real time by the weight-measuring part 100 and the joint angle-measuring part 200. The amplifier and the converter 500 may be included in the weight-measuring part 100 and the joint angle-measuring part 200, or may be formed in a separate portion. Also, the amplifier and the converter 500 may be included in the controller 300.

This rehabilitation apparatus using game device may be configured as a single user use so that a single user can play a game alone. Also, this rehabilitation apparatus using game device may be configured as a multi-user use so that at least two users can simultaneously play a game.

When this rehabilitation apparatus using game device is configured as the multi-user use, the personal game module 700 may be configured to be plural so that each of a plurality of game users can individually employ the personal game module 700. The controller 300 is connected to each personal game module 700 in a wired or wireless manner. The display part 400 may be provided for each user, or a plurality of users may share a single display part 400.

The controller 300 functions as a game server that provides a game environment so that the user can play a game with another user using the personal game module 700. All of users may play the same game, or each user may play a different type of a game. Meanwhile, even though FIG. 17 illustrates that a single controller 300 is provided to a plurality of personal game modules 700, a single controller 300 may be provided for each personal game module 700. Here, the respective controllers 300 may be directly connected to each other or may be connected via a server.

The game program mentioned in the invention indicates a game that the user may play alone or with another user using the personal game module 700. The game controllable by the controller 300 of the invention may be a game of sports, a strategic simulation game, a casual game, and the like in which at least one game element or game character manipulated by the controller 300 exists and control of a position or a movement of each character or a game element operated by a character acts as an important factor in a game circumstance. The game program may be configured to control a movement of a character according to rehabilitation exercise of the user based on the current game circumstance. Here, the game element operated by the character indicates a weapon or instrument used by the character, and the like. The simulation game is a game in which the user experiences a simulation circumstance similar to the reality on a video screen or a computer, and for example, there are a car driving game, an airplane flying game, a simulation battle game, and the like. Meanwhile, the casual game indicates a game that is operated with a simple game rule as compared to a strategic simulation game and the like requiring a long period of experience, a high game operation technology, and the like. An example of the casual game includes a puzzle game, a brickwork game, and the like. However, types of games controllable by the controller 300 according to the invention are not limited to the types of games described above. As long as the controller 300 can control a movement of a character according to the rehabilitation exercise of the user based on the game circumstance from the spirits of the invention, any game is included in the range of the invention.

A game controlled by the controller 300 according to the invention may include a game element configured as graphics, and a game space in which the graphic game element is configured to perform a predetermined game action.

The game element is an element constituting the game within the virtual game space, and indicates that the predetermined game action can be performed by the user. The game element may be configured into various types based on a type of a game being executed by the controller. For example, the game element may be a car manipulated by the user in a car racing game, may be a puzzle piece in a puzzle game, may be a cursor to select a predetermined character or a predetermined region of a game screen in a simulation game, and may be a character that fights in a martial arts game.

The game action performed by the game element indicates a predetermined action that is induced by a user's intent and thereby is performed by the game element in the game space. For example, the game action performed by the game element may be switching of a direction of a car, manipulated by the user, to left and right directions, or acceleration and deceleration of the car in the car racing game, may be a movement of a puzzle piece to a predetermined direction or direction switching of the puzzle piece in the puzzle game, may be a movement of a cursor to a predetermined direction or a selection of a predetermined character or a menu in the simulation game, and may be a movement of a fighting character, or hitting or kicking of the character in the martial arts game.

In the case of the rehabilitation apparatus using game device of the invention, weight data measured by the weight-measuring part 100 and joint angle data measured by the joint angle-measuring part 200 may act as a command that is input for the game action of the game element. That is, the weight data may act as a first command that is input for a first game action of the game element, and the joint angle data may act as a second command that is input for a second game action of the game element. Therefore, the game element of the game configured by the controller 300 may perform at least one game action according to the rehabilitation action of the user. Various complex rehabilitation actions of the user may act as commands of various game actions of the game element. A predetermined game action of the game element to be performed with respect to a predetermined command is stored in the controller so that the game element may perform the predetermined game action if the predetermined command is input from the user. The game element of the game may perform the predetermined game action according to the command by changing the weight of the user or the joint angle.

As another embodiment of the invention, the controller may include a module to calculate a motion amount of the user using data measured while a video game is in progress, and to display the motion amount of the user on a display part as a result of the video game or a result of a rehabilitation exercise based on the calculated motion amount.

Hereinafter, a detailed embodiment of the game mentioned in the invention will be described.

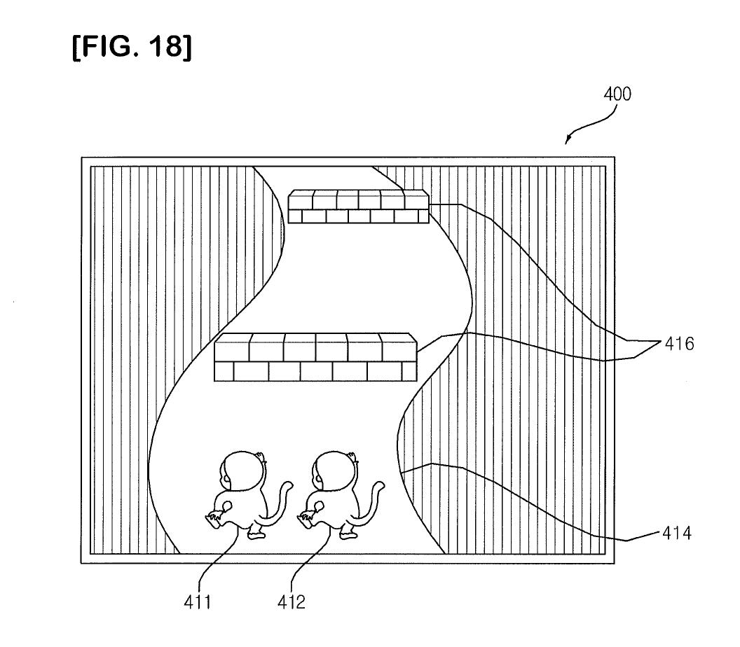

FIG. 18 illustrates a screen of a display part on which a game is displayed to describe an example of a game executable using the invention. The game illustrated in FIG. 18 is a game of jumping over an obstacle while moving along a predetermined route in a virtual track.

A character 411 manipulated by the user in the virtual game space, a character 412 manipulated by another user or a computer, a track 414 to be run by the character, and obstacles 430 to be jumped over by the character are displayed on a screen of the display part 400. The user may move the character 411 on the screen to left and right by moving the weight to left and right, and may continue the movement of the character 411 to the left and right direction by maintaining a status in which the weight is moved. Here, a distance moved to left and right at one time by the character 411 is in proportion to the magnitude weight-loaded on the leg. Movement of the character to one direction continues in proportion to a time of maintaining the weight on one leg. Also, the user may enable the character 411 to jump over the obstacle by changing the angle of a joint. Here, a jump height of the character 411 is in proportion to a change amount in the angle of the joint of the user. That is, in this game, the game element is the character 411 manipulated by the user, the game action of the game element performed by the user's intent is the movement of the character 411 into left and right direction, maintaining of the movement into one direction, and jump. Based on a game setting, the character 411 may be configured to perform a game action of pushing or holding the other character 412 in order to interrupt run of the other character 412. Such game action may be set to be configured through a combination of a weight movement of the user and a change in the angle of the joint.

When the track 414 on the screen of the display part 400 approaches a user side, that is, when the screen is scrolled down, the user moves the character 411 to left and right by moving the weight to left or right along a curved direction of the track 414 to prevent the character 411 from getting out of the track 414. Also, the user enables the character 411 to jump over the obstacle 416 by changing the angle of the joint. When the character 411 gets out of the track 414 or does not jump over the obstacle 416, advance of the character 411 may be set to be delayed or to restart from a start line.

When the user plays a game alone, the game may progress according to a track finish time reducing scheme. When a plurality of users plays the same game, the game may progress according to a ranking competition scheme.

A game similar to Tetris may be used as another embodiment of the game configurable by the rehabilitation apparatus using game device of the invention.

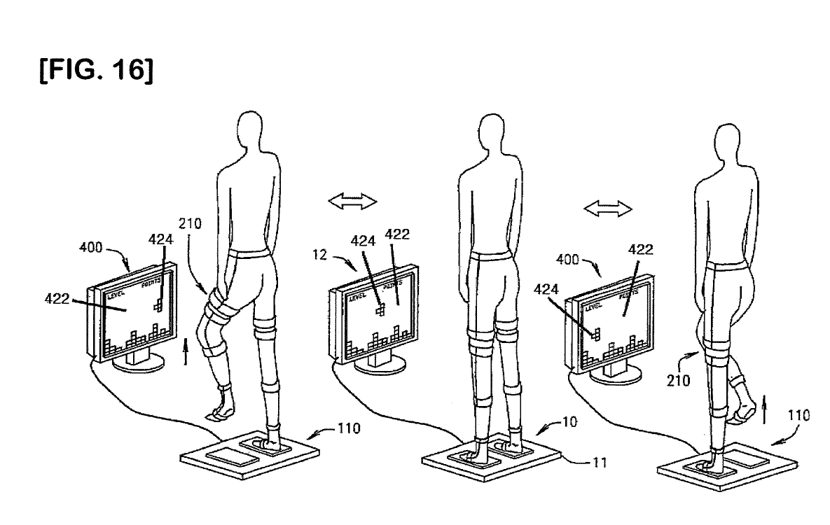

Referring to FIG. 16, the game similar to Tetris (hereinafter, “brickwork game”) is targeted to build up bricks so that no gap may occur between adjacent bricks. A space 422 in which bricks 424 can be built up is displayed on the screen of the display part 400. The brick 424 may be configured to move to left and right directions, or to rotate clockwise or anticlockwise. In this game, the game element is the brick 424, and the game action of the game element is a movement of the brick 424 into left and right direction and clockwise or anticlockwise rotation of the brick 424.

When the brick 424 starts to come down from the top of the space 422 of the game screen, the user determines a position and a direction in the space 422 to place the brick 424. A method of moving the brick 424 from a current position to left and right directions may be configured to move the brick 424 to left if the user moves the weight to the left lower limb, and to move the brick 424 to right if the user moves the weight to the right lower limb. Also, a method of rotating the direction of the brick 424 may be configured to rotate the brick 424 by changing the joint angle of partially paralyzed lower limb. The game continues in such a manner that bricks completely built up without any gap are removed when the bricks 424 are built up without any gap. However, the game is terminated if layers with a gap increase and the space 422 in which bricks can be built up is filled up.

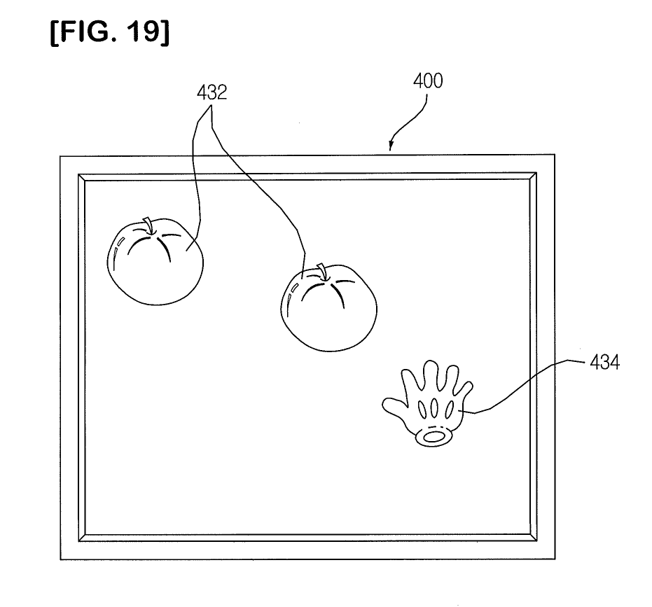

FIG. 19 shows a fruit picking game as still another embodiment of the game configurable by the rehabilitation apparatus using game device of the invention. At least one fruit 432 and a cursor 434 of a hand shape are displayed on the screen of the display part 400. The cursor 434 moves to left and right by movement of the weight to left and right, and the cursor 434 moves to up and down by reduction and expansion in the joint angle of the lower limb. If a predetermined time passes after the cursor 434 moves by an action of the user and thereby contacts with the fruit 432, the fruit 432 disappears and scores raise up. As the game performance ability of a patient is improved, a fruit displayed on the screen is changed to a smaller size fruit in order to increase a training difficulty. For example, the fruit is changed from a watermelon to an apple. In addition to the track game and the brickwork game that are used as examples to describe the game configurable by the rehabilitation apparatus using game device of the invention, various types of games may be configured by the rehabilitation apparatus using game device of the invention. When the user performs leg training through the rehabilitation apparatus using game device of the invention, the user may be interested and be immersed in the training with unflagging interest.

Also, data obtained through the game may be used to evaluate the lower-limb rehabilitation training performance of the user. For example, in the track game, the number of failures with respect to obstacle pass, the number of track leaves, a track finish time, and the like may be evaluation criteria of the rehabilitation training. In the brickwork game, a game continuance time, the number of built-up bricks, and the like may be evaluation criteria of the rehabilitation training.

A reference will now be given to an embodiment of a method for lower-limb rehabilitation training using the apparatus for lower-limb rehabilitation training based upon weight load and joint angle.

The invention proposes lower limb rehabilitation training in which the user performs task actions once or repeatedly to recognize the usage of the paralyzed muscle via the apparatus for lower limb rehabilitation training. Furthermore, in order to improve the recognized condition of paralysis, the user repeatedly corrects the task actions or the task actions are continuously changed so that the user carries out the varying task actions.

Examples of the task actions may include bending the knee of one lower limb with the weight loaded on the lower limb by a maximum amount, simultaneously bending the knees of both lower limbs with feet soles touching the floor, raising the heels of both feet with their toes touching the floor and walking in the same place. These actions are examples of the method for lower limb rehabilitation training, but the task actions may include any actions that can improve the usage of the paralyzed muscle of the user. Furthermore, the task actions can be corrected based upon the usage of the paralyzed muscle of the user.

In case that the task action is to bend the knee of one lower limb with the weight loaded on the lower limb by a maximum amount, the user can enhance muscular power for loading more weight to the paralyzed leg as well as for more bending the knee of the paralyzed leg while performing the task action. As a result, it is possible to improve the problem of a patient with partial paralysis that the patient can hardly support the weight with the paralyzed lower limb while bending the knee thereof.

Furthermore, as described above, the lower limb rehabilitation training may adopt a method for varying task actions so that the user can train all muscles or motors necessary for training while performing the varying task actions. The varying task actions may be displayed in such a fashion that the user can perform actions along with a varying graph or by letters or a display unit such as LED. These are examples only, but any method for allowing the user to recognize the task actions as soon as possible can be apparently adopted without departing from the purpose of the invention.

The task actions illustrated as above will now be described in more detail.

Balance Training

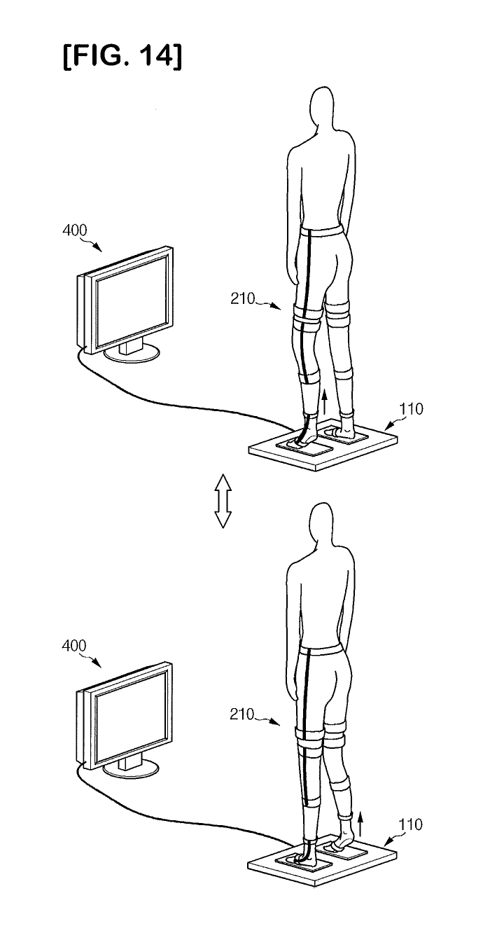

FIG. 14 illustrates a balance training of a user. The balance training can be performed in such a way that the user raises heels alternatively with toes of both lower limbs touching the floor in order to load weight on the paralyzed lower-limb.

Before performing the balance training, an apparatus for lower-limb rehabilitation training utilizing weight load and joint angle is attached to the user. In the apparatus for lower-limb rehabilitation training adapted to measure joint angle, the joint angle-measuring part 200 may include at least one of the knee joint goniometer, the ankle joint goniometer and the hip joint goniometer 202.

As shown in FIG. 14, the balance training repeats alternatively raising the heels with the toes touching the floor, in which one foot is lowered with total sole of the foot touching the floor or the weight-measuring part 100 while the heel of the other foot is raised with toes thereof touching the floor.

For example, it is assumed that the user performing the balance training is a patient with paralysis in right lower limb. The apparatus for lower-limb rehabilitation is attached to the patient with lower-limb paralysis, and then the patient starts training. It is also assumed that the joint angle-measuring part 200 of the apparatus for lower-limb rehabilitation includes all of the knee, ankle and hip joint goniometers. While the reference data necessary for the user may be generated previously by adopting a step of inputting the weight, height, age and so on of the user before the start of training, it is assumed in the following description that the normal left lower limb is measured at the same time to generate the data of the left lower limb as the reference data.

In case of setting the reference data by the data measured from the normal lower limb, the heels of the normal and paralyzed lower limbs are alternatively raised. So, in order to compare the conditions of the normal and paralyzed lower limbs with the heels raised, those data measured at the same time are not compared with each other. Instead, one is selected from the measurement data and the reference data, and the value of the selected data, which is taken half-period before the present time, is compared with the present value of the unselected data.

As the user raises the heel of the left lower limb, the angle of the ankle becomes larger while the angle of the knee becomes smaller. Since the leg is raised merely slightly, the angle of the hip becomes larger from about 180°. Also, the weight is biased to the right lower limb, and the center of the weight also moves toward the right lower limb. When the heel of the left lower limb is dropped with the whole part of the foot sole of the left lower limb touching the floor and the heel of the right lower limb is raised, the weight biased on the right lower limb moves to the left lower limb and the joint angle of the right tower limb changes. As assumed above, compared to the left lower limb, raising the heel of the paralyzed right lower limb will show differences in the degree of raising the lower limb, the degree of bending the knee and the degree of reducing the angle of the ankle. In addition, the time necessary for raising the heel of the paralyzed lower limb will be different from the time necessary for raising the heel of the normal lower limb. Therefore, following estimation can be made to the measurement data including weight data, joint angle data and time data; and the reference data including weight data, joint angle data and time data.

Provided that the paralyzed right lower limb has a smaller force for raising the leg than the left lower limb, the degree of biasing of the weight from the right leg to the left leg will be small. In addition, the paralysis of the right lower limb will make it difficult bend its knee as well as dorsiflex its ankle. Accordingly, it is assumed that the weight data of the measurement data (i.e., the measurement value on the right lower limb) will be larger than the weight data of the measurement data (i.e., the measurement value on the left lower limb) and any measurement data obtained by the comparison of the two weight data will be beyond an error range of weight. It is also assumed that the knee angle data of the measurement data will be smaller than the knee angle data of the reference data and any knee angle comparison data generated through the two knee angle data will be beyond an error range of knee angle. In addition, the ankle angle data of the measurement data will be smaller than the ankle angle data of the reference data and the hip angle data of the measurement data will be smaller than the hip angle data of the reference data. Provided that any condition data obtained through the respective comparison data are beyond a condition error range, there is generated a message instructing that the weight be further loaded on the left lower limb according to the importance of the weight data of the reference data and the weight data of the measurement data. The message is displayed on the display part 400, included in result data.

When the user refers to the message, if the condition data is beyond the error range but the weight data is within the error range, the user detects whether or not the knee angle comparison data is within the error range. Since it is assumed that the knee angle comparison data is beyond the error range and the knee angle data of the measurement data is smaller than the knee angle data of the reference data, the controller 300 of the apparatus for lower-limb rehabilitation training generates a message instructing that the knee of the right lower limb be further bent. The message is displayed on the display part 400, included in result data.

If the condition data is out of the error range but the weight and knee angle comparison data are within the error range, detection is made to the importance of the knee angle comparison data and the hip angle comparison data. Provided that the hip angle comparison data is larger than the hip angle comparison data owing to weak force of the user for raising the lower limb, there is generated a message instructing that the right lower limb be further raised according to the importance of the hip angle data of the measurement data and the angle data of the reference data. The message is then included in the result data.

Furthermore, the time spent for raising the heel of the right lower limb will be longer than the time spent for raising the heel of the left lower limb. In response to it, through the measured and reference time data, there is generated a message instructing that the right lower limb be more positively used.

Through the balance training as described above, the patient with lower-limb paralysis can have training to use paralyzed muscle by loading the weight to the paralyzed lower limb.

Standing

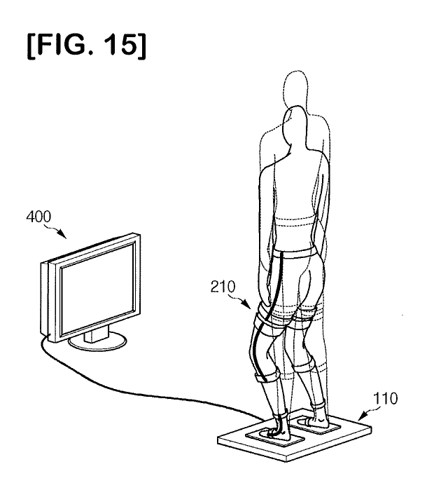

FIG. 15 illustrates a standing training of a user. In the standing training, the user with the apparatus for lower-limb rehabilitation training utilizing weight load and joint angle attached thereto repeats bending and straightening both knees. While bending and straightening both knees, the user trainings to bend the both knees simultaneously to the same angle within the same time period.

The above-mentioned balance training can utilize normal person's data previously stored in the apparatus for lower-limb rehabilitation training or data measured from a normal lower limb as the reference data to be compared with the measurement data of the paralyzed lower limb. In case of utilizing the data measured from a normal lower limb, the lower limb has the same weight load and joint angle. Unlike the balance training, the standing training utilizes simultaneously measured data in the comparison of the reference data with the measurement data.

Where a patient having paralyzed right lower limb takes a balance training, the apparatus for lower-limb rehabilitation is attached to the user so that the user starts the training. Before starting the training, basic information of the user can be inputted. While the basic user information is used for the identification of the user, it may also be used as data for setting the reference data in case of using the previously stored normal person's data. Through the standing training using the apparatus for lower-limb rehabilitation training proposed by the invention, the user can confirm whether or not the same weight load acts on both lower limbs. The user can also confirm whether or not the knee, ankle or hip angle is identical in the both limbs. As a result, in case of an abnormal standing posture where the user bends knees slightly while loading the weight similarly to both of the lower limbs, the training can help the user have a normal standing posture. That is, the user can bend the knees normally.

Walking

FIG. 16 illustrates a walking training of a user in a stepwise fashion.

A normal walking is carried out by organic movement of the joints of the lower limbs together with the weight load. Describing this in more detail, the walking training of this invention means that the user repeats walking in the same place. As a normal walking in the same place, when the total weight is loaded on the first lower limb, the user raises the second lower limb with its knee bent. Then, the user lowers the raised lower limb down, and at the same time, raises the first lower limb with the foot sole touching the floor and bends the knee of the first lower limb. These actions are repeatedly performed.

In the walking training, it is preferable to compare both lower limbs with each other about the condition where the leg is raised, as in the balance training. In case that the measurement data of the normal lower limb is used as the reference data, in order to generate weight comparison data, joint angle comparison data and so on, one is selected from the measurement data and the reference data and the value of the selected data, which is taken half-period before the present time, is compared with the present value of the unselected data.

However, a patient with lower-limb paralysis is not likely to bend the knee or raise the leg of the paralyzed lower limb, and thus will not bend the knee or raise the leg of the paralyzed lower limb as much as the normal lower limb. Accordingly, in order to correct the abnormal walking of the patient with lower-limb paralysis to the normal walking, it is important to provide the angle-measuring parts for measuring changes in the angle of the knee joint and the hip joint.

With the use of the apparatus for lower-limb rehabilitation training of the invention, the user can take a walking training by confirming whether or not the weight load and the joint angle change show normal conditions.

As described hereinbefore, the apparatus and method for lower-limb rehabilitation training of the invention is provided to detect the weight loaded on lower limbs, measure the angle of at least one joint of the knee, ankle and hip, and analyze the usage of the paralyzed muscle of the user through the measurement data. Therefore, the invention can improve the behavior of patients with lower-limb paralysis who tend to move without using a joint, and thus the patients can take an training of moving the joint and the weight load systematically.

Furthermore, the invention can combine a game into a lower-limb rehabilitation training to stimulate the user with interest in the training, thereby inducing the user to perform repeated actions without getting bored.

In the apparatus and method for rehabilitation training of patients with lower-limb paralysis of the invention, the advantages of the invention is that the usage of paralyzed muscles was evaluated more precisely based upon the movement of the weight load and the angle of the joint and the user can correct the standing posture and train muscles necessary for walking.

Since the invention enables measurement and rehabilitation training for partial paralysis to be conducted two-dimensionally, there are advantages in that a large space or expensive equipments are not needed. Furthermore, since it can be enabled by a simple system, real-time feedback can be afforded to the user.

The invention also displays measurement and analysis results of usage of the paralyzed muscle on graphs so that the user can easily recognize the usage of the paralyzed muscle. In addition, the invention provides games using the measurement and analysis results as variables in order to stimulate the user with interest in training.

The invention can also advantageously allow the user to recognize muscles or motors, which are not unused owing to paralysis, while the user repeatedly performs the task actions so that the user can improve the usage of the paralyzed muscle.

The apparatus and method for lower limb rehabilitation training of the invention is designed for patients for partial paralysis, and as apparent in view of the purpose of the invention, can be applied to patients with paralysis in both lower limbs or those having poor balancing capability without paralysis. It should also be construed that the invention can be modified without departing from the purpose or scope of the invention.

While the present invention has been shown and described in connection with the preferred embodiments, it will be apparent to those skilled in the art that modifications and variations can be made without departing from the spirit and scope of the invention as defined by the appended claims.

What is claimed is:

1. A rehabilitation apparatus using game device comprising:
a display part to display, for a user, a video game including a graphical game element and a game space in which the graphical game element performs at least one game action by a command;
a personal game module comprising a force plate being placed below the lower limb to contact with the bottom surface of the lower limb and to measure a value of pressure applied by the lower limb and thereby generate measured weight data, and a joint angle-measuring part to measure a joint angle value of at least one joint of the lower limb and thereby generate measured joint angle data, and to generate the command for the game action of the game element; and
a controller configured to generate the video game, the controller being configured to control a first game action of the graphical game element of the video game based on a change in the measured value of pressure, and to control a second game action of the graphical game element of the video game based on a change in the measured joint angle value of the lower limb.

2. The rehabilitation apparatus using game device of claim 1, wherein the joint angle-measuring part includes an electric goniometer attached to at least one joint, and includes at least one of a knee joint angle-measuring part for measuring the angle of a knee joint, an ankle joint angle-measuring part for measuring the angle of an ankle joint, and a hip joint angle-measuring part for measuring the angle of a hip joint.

3. The rehabilitation apparatus using game device of claim 2, wherein the joint angle-measuring part includes all of the knee joint goniometer, the ankle joint goniometer, and the hip joint goniometer in order to synthetically measure whether the lower limb is moved using major muscles of the lower limb including thigh muscles and calf muscles, and whether there is a force for raising the lower limb.

4. The rehabilitation apparatus using game device of claim 1, wherein the joint angle-measuring part includes at least one of a tilting sensor or a gyro sensor attached to the lower leg or the thigh, an accelerometer attached to a knee joint, a magnetic sensor for measuring a distance between a reference point and a predetermined region of a body of the user, and an ultrasonic distance measuring device, and indirectly measures a reduction and an expansion in the joint angle of the lower limb.

5. The rehabilitation apparatus using game device of claim 1, wherein the first game action is a movement of the graphical game element into one direction and a movement into an opposite direction thereto in the game space.

6. The rehabilitation apparatus using game device of claim 1, wherein:
at least two personal game modules are provided,
each personal game module is connected to a single controller, and
each of at least two users is enabled to play the same game by individually employing the personal game module.

7. A rehabilitation apparatus using game device comprising:
a display part to display, for a user, a video game including a graphical game element and a game space in which the graphical game element is configured to perform at least one game action by a command input from the outside;
a personal game module including a force plate being placed below the lower limb to contact with the bottom surface of the lower limb and to measure a value of pressure applied by the lower limb and thereby generate measured weight data, and an angle-measuring part to measure an angle value of the lower limb and thereby generate measured angle data, and to input the command for the game action of the game element; and
a controller to generate the video game required to vary the weight applied to the lower limb and the joint angle of the lower limb, to control a first game action of the graphical game element of the video game based on a change in the measured value of pressure, and to control a second game action of the graphical game element of the video game based on a changed in the measured angle value of the lower limb.

8. The rehabilitation apparatus using game device of claim 7, wherein the angle-measuring part includes an electric goniometer attached to at least one joint, and includes at least one of a knee joint angle-measuring part for measuring the angle of a knee joint, an ankle joint angle-measuring part for measuring the angle of an ankle joint, and a hip joint angle-measuring part for measuring the angle of a hip joint.

9. The rehabilitation apparatus using game device of claim 8, wherein the angle-measuring part includes all of the knee joint angle-measuring part, the ankle joint angle-measuring part, and the hip joint angle-measuring part in order to synthetically measure whether the lower limb is moved using major muscles of the lower limb including thigh muscles and calf muscles, and whether there is a force for raising the lower limb.

10. The rehabilitation apparatus using game device of claim 7, wherein the angle-measuring part includes at least one of a tilting sensor or a gyro sensor attached to the lower leg or the thigh, an accelerometer attached to a knee joint, a magnetic sensor for measuring a distance between a reference point and a predetermined region of a body of the user, and an ultrasonic distance measuring device, and indirectly measures a reduction and an expansion in the joint angle of the lower limb.

11. The rehabilitation apparatus using game device of claim 7, wherein the first game action is a movement of the graphical game element into one direction and a movement into an opposite direction thereto in the game space.

12. The rehabilitation apparatus using game device of claim 7, wherein:
at least two personal game modules are provided,
each personal game module is connected to a single controller, and
each of at least two users is enabled to play the same game by individually employing the personal game module.

13. A rehabilitation apparatus using game device comprising:
a display means for displaying, for a user, a video game including at least one graphical game element and a game space in which the graphical game element is configured to perform at least one game action by a command;
a weight-measuring part being placed below the lower limb to contact with the bottom surface of the lower limb and to measure a value of pressure applied by the lower limb and thereby generate measured weight data;
a joint angle-measuring part to directly measure a joint angle value of at least one joint of a knee joint, an ankle joint, and a hip joint of the lower limb or indirectly measure the joint angle of the lower limb and thereby generate measured joint angle data; and
a controller configured to generate the video game, the controller being configured to control a first game action of a first graphical game element of the video game based on a change in the measured value of pressure, and to control a second game action of a second graphical game element of the video game based on a change in the measured joint angle value.

14. The rehabilitation apparatus using game device of claim 13, wherein the joint angle-measuring part includes at least one of a knee joint angle-measuring part for measuring the angle of a knee joint, an ankle joint angle-measuring part for measuring the angle of an ankle joint, and a hip joint angle-measuring part for measuring the angle of a hip joint.

15. The rehabilitation apparatus using game device of claim 13, wherein the first graphic game element and the second graphic game element are the same game element, and the first game action and the second game action are different from each other.

16. The rehabilitation apparatus using game device of claim 13, wherein the first graphic game element is a character and the second graphic game element is a game element operated by the character.

17. The rehabilitation apparatus using game device of claim 13, wherein the first game action is a movement of the graphical game element into one direction and a movement into an opposite direction thereto in the game space.

18. The rehabilitation apparatus using game device of claim 13, wherein the first game action adjusts a size of the graphic game element in the game space.

19. The rehabilitation apparatus using game device of claim 13, wherein the controller generates movement amount data of the user as a result of the video game to display the movement amount data on the display part.

20. The rehabilitation apparatus using game device of claim 13, wherein the joint angle-measuring part includes at least one of a tilting sensor or a gyro sensor attached to the lower leg or the thigh, an accelerometer attached to a knee joint, a magnetic sensor for measuring a distance between a reference point and a predetermined region of a body of the user, and an ultrasonic distance measuring device, and indirectly measures a reduction and an expansion in the joint angle of the lower limb.
